(19) United States
(12) Patent Application Publication
(10) Pub. No.: US 2023/0041478 A1
(43) Pub. Date: Feb. 9, 2023

(54) A CONVEYING DEVICE FOR CONVEYING MEDICAL FLUIDS THROUGH A HOSE

(71) Applicant: Fresenius Medical Care Deutschland GmbH, Bad Homburg (DE)

(72) Inventors: Gerhard Spengler, Euerbach (DE); Udo Kirstgen, Rottenburg (DE)

(21) Appl. No.: 17/784,220

(22) PCT Filed: Dec. 10, 2020
(86) PCT No.: PCT/EP2020/085589

§ 371 Date: Jun. 10, 2022

(30) Foreign Application Priority Data
Dec. 11, 2019 (DE) 10 2019 133 969.3

(51) Int. Cl. A61M 60/279; A61M 60/462; A61M 60/113; A61M 60/845; A61M 60/37

(52) U.S. Cl. CPC A61M 60/279; A61M 60/462; A61M 60/113; A61M 60/845; A61M 60/37

(57) ABSTRACT

A conveying device for conveying a medical fluid that is guided into a hose includes a rotor and a stator. The stator includes a stator base, an area for receiving the hose in the conveying device, and a hose bed providing a counter bearing for occlusion devices. The rotor includes a rotor axis and at least two occlusion devices that are attached radially to the rotor axis and that compress the hose intermittently against the hose bed during use of the conveying device. The rotor encompasses an axial guiding element that is arranged to rotate about its own longitudinal axis for aligning the hose in the stator. The longitudinal axis of the axial guiding element extends parallel to the longitudinal axis of the rotor axis. The axial guiding element includes a middle section and a first lateral section protruding radially over the middle section.

### CROSS-REFERENCE TO RELATED APPLICATIONS

The present application is the national stage entry of International Patent Application No. PCT/EP2020/085589, filed on Dec. 10, 2020, and claims priority to Application No. DE 10 2019 133 969.3, filed in the Federal Republic of Germany on Dec. 11, 2019, the disclosures of which are expressly incorporated herein in its entirety by reference thereto.

### TECHNICAL FIELD

The present disclosure relates to a medical conveying device for conveying a fluid guided in a hose. It further relates to a blood treatment apparatus.

### BACKGROUND

Hose pumps or peristaltic pumps for conveying a fluid which is guided in a hose are known from medical technology practice. Hose pumps are a special form of positive displacement pumps in which the working space consists of the lumen of a flexible hose. The working space is changed by externally induced mechanical deformation using occlusion devices (for example in the form of occlusion rollers or surfaces). To generate a volume flow, the hose is occluded at one or more points (occlusion area) and this occlusion area is moved forward along the hose towards the pump outlet. The suction of new fluid at the pump inlet is done by negative pressure due, amongst others, to the reshaping of the now no longer occluded hose area due to the elasticity of the hose.

It is an object of the present disclosure to propose a further conveying device. In addition, a blood treatment apparatus is to be specified.

The object according to the present disclosure is achieved by a conveying device having the features of claim 1. It is further achieved by a blood treatment apparatus.

The conveying device according to the present disclosure serves for conveying a medical fluid guided in a hose. The fluid may be or may encompass whole blood, plasma or other blood components, pharmaceuticals, substituate, dialysate, parenteral food, or other medical fluid.

### SUMMARY

The conveying device according to the present disclosure includes a rotor with occlusion devices and a stator. The stator may, for example, be the pump housing or part thereof. It forms or includes a receptacle for the rotor and the hose. Thus, the stator includes at least one stator base, a hose bed and a space for receiving the hose, wherein the hose bed is or includes a counter bearing for the occlusion devices of the rotor. The stator may be accessible or open, for example, in a direction of the axis of rotation of the rotor, or may include a closable and/or removable cover element which protects the rotor and the hose, amongst others, from accidental contact. The stator further encompasses an inlet area and an outlet area for the hose, which may be designed for example as pump inlet and pump outlet. The conveying device sucks medical fluids from the inlet area and conveys them further to the outlet area.

The rotor encompasses at least one rotor axis (which may be designed for example as geometric axis of a hub bore or may herein be alternatively a rotor shaft instead of an axis), such as that arranged centrally (for example relative to the rotor), and at least including two occlusion devices, for example in the form of rollers. The occlusion devices, which are mounted radially with respect to the rotor axis, serve, when the conveying device is in use, to compress the hose intermittently against the counter bearing of the stator and to press or convey the fluids in the hose lumen towards the pump outlet. This creates a negative pressure in the hose upstream of the occlusion area, which sucks further fluid to be conveyed.

The hose is compressed alternately and can decompress again. The rotor is not equipped with occlusion devices over its entire circumference, rather these are arranged with gaps that allow the hose to be intermittently occluded while the rotor is rotating. Such occlusion devices may be present in different numbers. For example, they may be offset from each other by 180° for rotors with two occlusion devices, by 120° for rotors with three occlusion devices, by 90° for rotors with four occlusion devices, etc. The distance between the occlusion devices is the same in some embodiments but not in others.

According to the present disclosure, the rotor also includes at least one guiding element which is arranged so as to be rotatable about its longitudinal axis and is referred to herein as the axial guiding element for the hose. It serves to align and/or center the hose in the hose bed of the stator. The longitudinal axis of the axial guiding element preferably extends parallel to the longitudinal axis of the rotor axis.

Since the axial guiding element may rotate about its longitudinal axis according to the present disclosure, it is preferably used to reduce the frictional forces acting on the hose and generated by the rotational movement of the rotor.

The rotary movements of the axial guiding element may be passive, i.e. without an own drive, i.e. only due to the frictional forces against the hose.

In certain embodiments, the same number of axial guiding elements may be provided as the number of occlusion devices that the rotor includes. Thus, with the rotor running, an axial guiding element may precede each occlusion device.

In further embodiments, the number of occlusion devices does not correspond to the number of axial guiding elements. For example, two or more axial guiding elements may be provided preceding each occlusion device.

For this purpose, the axial guiding element includes at least one middle (alternatively: central) section. This may be rotatably arranged or mounted. The axial guiding element also includes at least one first lateral section which is protruding radially over the middle section and rotatably arranged. The first lateral section may project radially beyond a section of an outer edge of the rotor.

The lateral section and the middle section of the axial guiding element receive the hose by delimiting it from below (in a side view of the rotor with its axis of rotation upright) and on the radially inner side of the hose.

Lateral sections and middle sections may in some embodiments be a one-piece component. They may alternatively be multi-part or assembled into a unit.

The blood treatment apparatus according to the present disclosure includes a conveying device. The conveying device may be part of the blood treatment device, or may be designed as a cassette or a module which is or will be attached to the blood treatment apparatus, for example at or in a receptacle of the blood treatment apparatus provided for this purpose, for example with a gear to which the rotor of the conveying device is, or may be, connected.

In all of the following statements, the use of the expression “may be” or “may have” and so on, is to be understood synonymously with “preferably is” or “preferably has,” and so on, respectively, and is intended to illustrate embodiments according to the present disclosure.

Embodiments according to the present disclosure may include one or several of the features mentioned supra or in the following. In this, the features mentioned herein may in any combination be subject-matter of embodiments according to the present disclosure, unless the person skilled in the art recognizes a specific combination as being technically impossible.

Embodiments according to the present disclosure are further subject-matter of the dependent claims and embodiments.

Whenever numerical words are mentioned herein, the person skilled in the art shall recognize or understand them as indications of a numerical lower limit. Unless it leads the person skilled in the art to an evident contradiction, the person skilled in the art shall comprehend the specification for example of “one” as encompassing “at least one”. This understanding is also equally encompassed by the present disclosure as the interpretation that a numerical word, for example, “one” may alternatively mean “exactly one”, wherever this is evidently technically possible for the person skilled in the art. Both are encompassed by the present disclosure and apply to all numerical words used herein.

Whenever an embodiment is mentioned herein, it is then an exemplary embodiment according to the present disclosure.

The information “top” and “bottom” are herein to be understood in case of doubt by the person skilled in the art as absolute or relative spatial information, which refer to the orientation of the respective element in the figures or to the orientation of same when used as intended.

If a bore is mentioned here, it may alternatively be an opening or a through-opening.

In some embodiments, the conveying device according to the present disclosure includes at least one further axial guiding element. In some embodiments, at least one of the axial guiding elements or at least one of the occlusion devices is arranged on an optional swing arm of the rotor.

In some embodiments the swing arm is designed as a suspension, for example, as a fork, with a swinging joint which connects the suspension or the fork to another section of the rotor, for example, a hub body.

A connecting element may be provided, by which the occlusion device is fastened to the suspension, wherein the occlusion device may be arranged freely rotating about this connecting element. The connecting element may form the axis of rotation of the occlusion device.

The optional hub body couples the swing arm and thus all the parts arranged on it to the axis of the rotor. In some embodiments, the hub body thus couples, for example, all components of the rotor that extend radially within or from the rotor, such as the occlusion devices and the axial guiding elements, to the rotation of the rotor.

In certain embodiments, the optional hub body is designed as a connecting plate, i.e. as a plate with the necessary bores, pins or other connection structures, in order to connect the rotor axis to the occlusion device(s) in such a way that the movement of the rotor axis is transmitted to the occlusion device(s).

In some embodiments, swing arms are designed as arms. They may then form rigid structures.

In some embodiments, at least one swing arm is spring-mounted. A low springiness of the swing arm(s) may, for example, be advantageous to facilitate the insertion of a hose into the rotor or to adapt the rotor to different hoses in a simple way.

In some embodiments, at least two axial guiding elements and/or at least two occlusion devices are arranged opposite to each other on a common swing arm of the rotor.

In this, the rotor may include one swing arm for the occlusion device(s) and one further swing arm for the axial guiding element(s).

Both the occlusion devices and the axial guiding elements may be present in different numbers. In their radial arrangement, they may be arranged on their respective swing arms at always the same distance or at different distances from each other.

In some embodiments, a plurality of occlusion devices and a plurality of axial guiding elements of the rotor are arranged on a common swing arm. Herewith an axial guiding element is optionally assigned to each occlusion device.

According to the present disclosure, the occlusion devices and axial guiding elements may be present in different numbers and may be arranged at the swing arm at the same or at different distances from each other.

In further embodiments, the number of the occlusion devices and the axial guiding elements is not the same. For example, two or more axial guiding elements may be arranged in the rotor preceding each occlusion device.

The swing arm may have all the designs known in the prior art.

In some embodiments, at least one of the axial guiding elements may include at least one second lateral section. This lateral section may be conical. The geometric design of the axial guiding elements serves for better guidance of the hose above the stator base and around the rotor in order to minimize the transverse sliding movements of the hose when the rotor is running and thus also to reduce the abrasion of the hose.

In a further embodiment, the axial guiding element includes a middle section that is slightly hourglass-shaped.

In some embodiments, the first and/or the second lateral section of the axial guiding element may be conical. Together, they may form a double cone, wherein the tips of the cones face each other and the middle section of the axial guiding element is arranged between, and connects, the first and the second lateral section.

The double-cone-shaped axial guiding element guides the hose around the rotor due to its geometric design. Further guiding elements, which would serve the same purpose, may advantageously be omitted in such embodiments according to the present disclosure.

In some embodiments, the two conical lateral sections are different or diverse from each other, which means that the double cone formed by the two conical lateral sections is preferably not point-symmetrical to its geometrical center of gravity.

In certain embodiments, the rotor includes axial guiding elements which, in addition to the middle section, include only exactly one lateral section, but not two lateral sections.

In some embodiments, the rotor may further include at least one lateral guiding element.

A longitudinal axis of the lateral guiding element advantageously extends perpendicular to the rotor axis and is arranged above the stator base of the stator, so that the hose is arranged between the stator base and the lateral guiding element. The lateral guiding element thus only delimits the hose to the outside, i.e. to the user. It may advantageously contribute to prevent the hose from sliding out of the stator base when the rotor is rotating.

In such embodiments, in addition to the middle section the axial guiding elements may only encompass exactly one lateral section toward the stator base, which prevents contact of the hose with the stator base. The middle section of the axial guiding element supports and guides the hose from below (i.e. it supports the surface of the hose which is arranged closest to the rotor axis). The lateral guiding element in turn prevents contact with the hub body of the rotor and prevents the hose from running outside the occlusion roller. In such embodiments, the hose is thus supported and guided to the outside, i.e. to the user, preferably exclusively, by one or more such lateral guiding elements.

In some embodiments, the lateral guiding element may be rotatable about its central axis. This may serve to reduce or minimize the mechanical stress for or on the inserted hose when the rotor is running.

In some embodiments, optionally only the middle section of the axial guiding element is rotatable about its central longitudinal axis. This may also help to reduce the mechanical stress on the hose.

In some embodiments, the conveying device according to the present disclosure is equipped with a cover element. The cover element may cover or at least partially cover the rotor and/or the hose, insofar as it is inserted into the conveying device.

In some embodiments, when the conveying device is conveying the cover element is arranged to be stationary relative to the stator and/or non-rotatable.

In further embodiments, the cover element is coupled to the rotor and is taken along by it when it rotates.

The cover element may be suitable for protecting the rotor or the entire conveying device from dirt or moisture. Furthermore, the user may be prevented for example, when the rotor is rotating, from accidentally reaching between components of which at least one is rotating.

In some embodiments, the cover element optionally includes, or consists of, at least one guiding lug. Due to its geometry, the guiding lug may be used for simple and safe insertion of the hose in the stator base.

The guiding lug may be designed, for example, as a lowering of a section of the cover element towards the stator base. When inserting the hose, it may be a supporting receptacle for that section of the hose which is to be inserted around the rotor.

When inserting the hose into the conveying device, the hose is placed in contact with the guiding lug. The hose is then advanced towards the stator base. The hose slides, for example, by a surface of the guiding lug being rounded, smooth and/or sloping in the direction of the stator base, to the desired position relative to the rotor and may then be advanced further around the rotor.

The geometry of the guiding lug preferably facilitates the insertion of the hose and prevents the hose from twisting and/or from slipping out of the space between rotor and stator during insertion.

In other embodiments, the guiding lug is integrated into the cover element and/or forms a one-piece unit with it.

In some embodiments, the guiding lug(s) and the cover element form a multi-piece unit.

In some embodiments, the guiding lug may be understood as an insertion or threading aid.

In some embodiments, optionally at least one axial guiding element and/or one lateral guiding element is integrated in the cover element.

In such embodiments, swing arms or similar connecting elements between the rotor and/or axial and/or lateral guiding elements are no longer necessary, since the task of these connecting elements is taken over by the cover element.

In some embodiments, the cover element is designed as a hub body. It may include a connecting element for its connection to the gear of the conveying device. It may also act as a swinging arm; for example, the cover element may be provided with pins which may serve as a shaft for the rotating components of the conveying device. Thus, it is advantageously possible to construct the conveying device without swing arms, hubs or similar connecting elements, since the connecting and coupling function which would normally be performed by the swing arm is here taken over by the cover element.

In some embodiments, the conveying device is a cassette or a part thereof, which is prepared to be attached to a fluid treatment apparatus.

In some embodiments, the conveying device is firmly connected to further sections of the fluid treatment apparatus, such as its housing.

In some embodiments, the fluid treatment apparatus is a blood treatment apparatus.

In some embodiments, the blood treatment apparatus is designed as a dialysis apparatus, hemodialysis apparatus, hemofiltration apparatus, hemodiafiltration apparatus, apheresis apparatus, plasma treatment or plasma exchange apparatus, TPE apparatus (total parenteral nutrition) and/or as combinations thereof.

In some embodiments, the distance between the occlusion devices and the hose bed being a counter-bearing for the occlusion devices is not variable.

In several embodiments, the hose bed does not have a conical or tapered footprint which is supported on or at the housing against a complementary shaped support surface.

In some embodiments, the distance between the occlusion devices and the hose bed is not adjustable by moving the hose bed relative to the housing along the support surface.

Some or all the embodiments may include one, several or all of the advantages mentioned below.

The conveying device according to the present disclosure allows, as a hose pump, to deliver fluids gently and without contact to components of the apparatus other than the inside of the hose.

The structure of the conveying device according to the present disclosure may also protect the hose from severe heavy or even damage by keeping low shear forces caused by the displacement of the occluded section of the hose along the longitudinal axis of the hose. Shear stresses are not only responsible for a reduced hose life, but also-with increasing hose wear-for a decrease in conveyance rate and conveyance pressure.

According to the present disclosure, a damage to the hose which may also advantageously be reduced is the so-called spallation of hose material particles. Such blown or chipped hose particles may contaminate the pumped fluid. In the medical field, it may for example lead to, their storage in organs.

The conveying device according to the present disclosure may advantageously reduce or even avoid the transverse movement of the hose (i.e. in a direction parallel to the axis of rotation of the rotating rotor) in the stator. This, in turn, indirectly reduces or diminishes abrasion of the hose due to sliding friction, thus increasing the life of the hose and reducing the phenomenon of spallation. This applies to the occluded hose section on the negative pressure side.

It may also be advantageous that when using the conveying device according to the present disclosure, such a favorable structure thereof not only significantly increases the life of the hose, but also allows more constant flow values to be maintained.

### BRIEF DESCRIPTION OF THE FIGURES

In the following, the present disclosure is exemplarily explained based on the accompanying drawings in which identical reference numerals refer to the same or to identical components. In the figures, the following applies:

FIG. 1 shows a schematic representation of a conventional conveying device;

FIG. 2a shows a schematic representation of a first embodiment of the conveying device according to the present disclosure in a front view;

FIG. 2b shows the schematic representation of the conveying device of FIG. 2a with an inserted hose;

FIG. 3 shows a schematic representation of the conveying device according to the present disclosure of FIG. 2a with a view from below on an occlusion device and on an axial guiding element;

FIG. 4 shows a schematic representation of the rotor of FIG. 2a in the view according to FIG. 3 during the insertion of a hose using the guiding lug;

FIG. 5 shows a schematic representation of the rotor of the preceding figures in the view of FIG. 4 with an inserted hose;

FIG. 6 shows a schematically simplified sectional view of the rotor of the preceding figures with an inserted hose;

FIG. 7 shows a perspective view of the rotor of the preceding figures obliquely from below and from the front on its hose guiding area;

FIG. 8 shows a further embodiment according to the present disclosure of the conveying device with a perspective view on the rotor obliquely from outside, with a view on an occlusion device, an axial guiding element in an embodiment having only one lateral section, as well as with one lateral guiding element;

FIG. 9 shows the rotor of FIG. 8 in a front view;

FIG. 10 shows a section of a further embodiment of the conveying device according to the present disclosure with a view on the rotor in a slightly perspective view from below;

FIG. 11 shows a schematic representation of a further embodiment of the conveying device according to the present disclosure in a front view without the cover element with a view on the swing arms; and

FIG. 12 shows a schematic representation of a further embodiment of the conveying device according to the present disclosure in a front view without the cover element with a view on the common axis of the swing arm and the axial guiding element.

### DETAILED DESCRIPTION

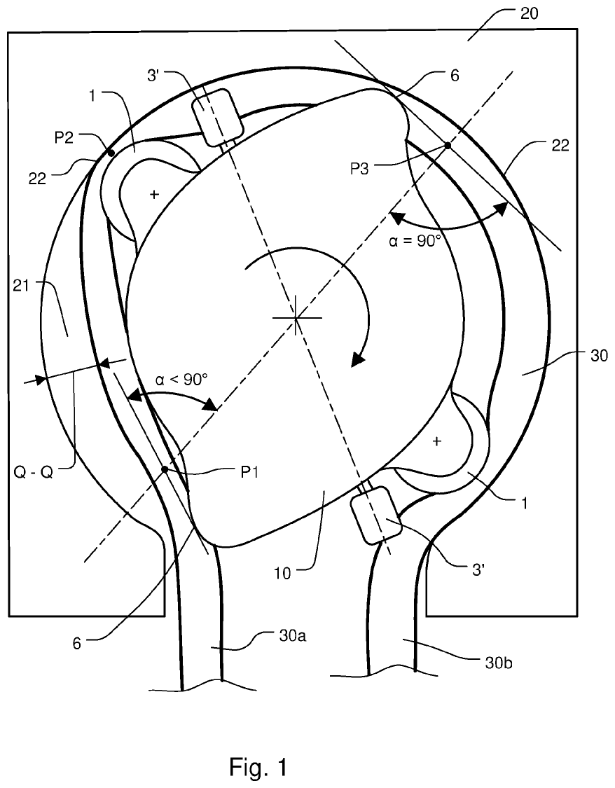

FIG. 1 shows a schematic representation of a conventional conveying device as a pump unit having a stator 20 and a rotor 10. The hose 30 is located above the stator base 21 in the space between rotor 10 and stator 20. The rotor 10 of FIG. 1 does not include any axial guiding elements being provided by the present disclosure.

With the rotor 10 running, transverse movements (see opposite arrows Q-Q) take place in the hose 30 relative to the occlusion rollers 1 or to the guiding element 6. The frictional forces that occur during the rotation of the rotor act on the hose 30 such that the latter is stretched like a string between the hose inlet and the occlusion roller 1 (see the hose course from point P1 to point P2).

In embodiments of the conveying device in which the hose 30 does not extend purely tangential at the inlet and the outlet of the pump housing (30a and 30b respectively) but rather slightly kinked, such transverse movements occur under the tension mentioned. The hose 30 is here cyclically tensioned with each complete turn of the rotor 10, wherein the tensile tension decreases again with further rotation of the occlusion device 1 towards the overpressure side. Transverse movements hardly take place on the overpressure side, since the hose 30 is only compressed there and lies against the hose bed.

With this string-like stretched hose section, the angle between the axis of the guiding surface and the hose axis is less than 90°, which in addition to causing the unrolling of the hose also causes the sliding of the hose over the roll. This effect promotes abrasion on the hose 30 in addition to the described transverse movement.

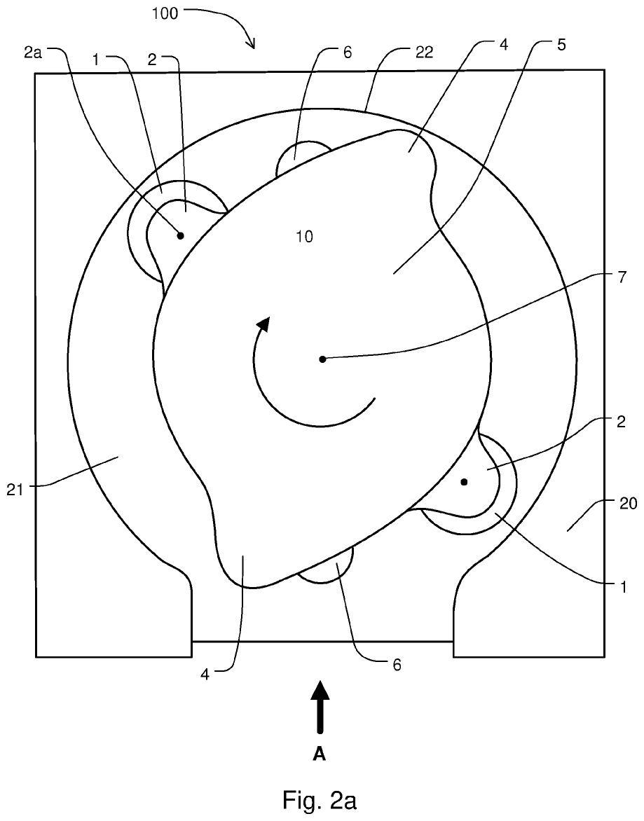

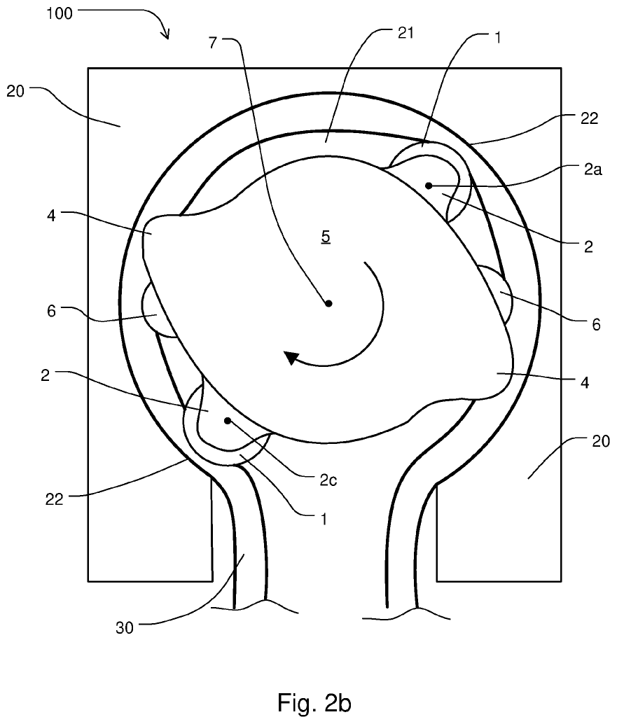

FIG. 2a and FIG. 2b both show a schematic representation of an embodiment of a conveying device 100 according to the present disclosure in a view from the front or from the outside (as perceived in use by the user) with a rotor 10 rotating about a rotor shaft 7 and with a fixed stator 20 in which the rotor 10 can rotate. The stator 20 forms a receptacle for the rotor 10 and for a hose 30 (see FIG. 2), which is inserted into the rotor 10 from below and exits from it again downwards.

The receptacle for rotor 10, which in FIGS. 2a and 2b lies behind the rotor 10, is either designed as part of a housing (not shown) of a fluid treatment apparatus or as a cassette, which is mounted or plugged on a rotor gear of the fluid treatment apparatus, not shown.

The rotor 10 may be connected via a shaft to the drive motor of the fluid treatment apparatus, not shown, and may be operated by said drive motor.

In certain embodiments, the receptacle for the rotor 10 may have, or may be, a drive shaft, and the rotor 10 is plugged onto it.

A receiving section of the stator 20 or a space for the hose 30 is formed by the stator base 21, from which a hose bed 22 extends as a counter bearing for the occlusion device 1. The hose 30 is inserted into the conveying device 100 such that it lies between the rotor 10 and the hose bed 22 as a counter bearing and is guided at least in sections around the rotor 10, as shown in FIG. 2b.

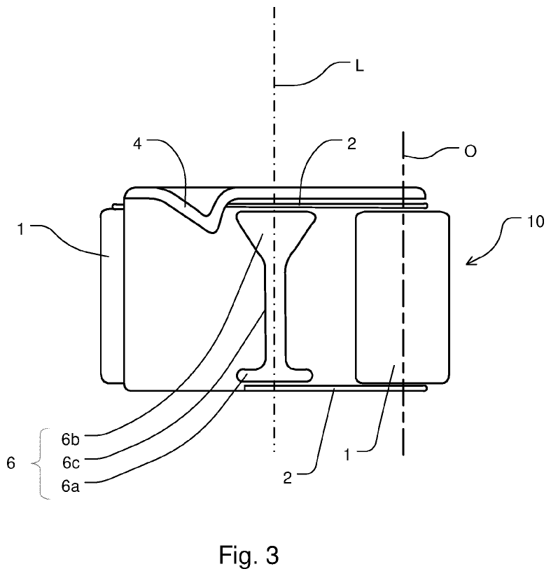

The rotor 10 includes one or more occlusion device(s) 1, which are, for example, designed as substantially cylindrical occlusion rollers and which may preferably rotate about their central axis of rotation O (see FIG. 3). FIG. 2a exemplarily shows two occlusion devices 1 which lie opposite to each other and are attached to a swing arm 2. The swing arm 2 connects the rotor shaft 7 with the occlusion devices 1 and couples them functionally to the rotating movement of the rotor 10. In this way, when the conveying device 100 is in use, the hose 30 is cyclically pressed by one or more occlusion devices 1 against the hose bed 22 as the counter bearing of the stator 20 and is thus occluded.

When the rotor 10 rotates, the occlusion devices 1 slide or roll along the hose 30 (not shown in FIG. 2a). After the occlusion devices 1 are disengaged from hose 30, the previously compressed section of the hose re-expands due to the elasticity of the hose material. The generated negative pressure sucks in the fluid present in the hose system upstream of the conveying device 100. The negative pressure is generated by the way in which the volume enclosed and present in front of the occlusion device 1 is conveyed. This also causes the fluid to be conveyed along the lumen of hose 30.

In this, the occlusion devices 1 may compress the hose 30 to a greater or lesser extent. The degree of this compression of the hose 30 may optionally be set according to the present disclosure. For example, it may be fixed by an optional, preferably spring-loaded swing arm 2.

By such an optional swing arm 2, the conveying device 100 may be adapted to the characteristics of the hose 30 actually used, for example, to its material properties or its cross section.

Furthermore, the conveying device 100 of FIG. 2a and FIG. 2b further includes at least one axial guiding element 6 (see FIG. 3), here exemplarily two axial guiding elements 6, of which a first lateral section may be seen in each case, see FIG. 3.

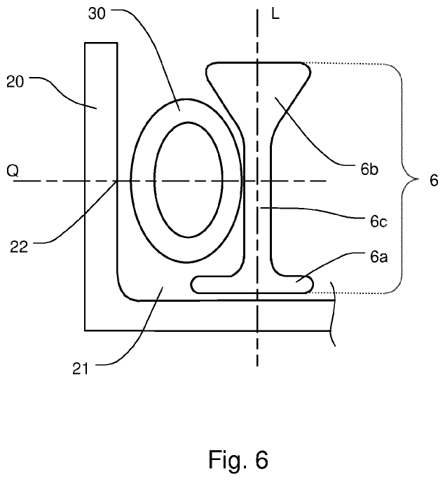

The axial guiding elements 6 favor an embedding of the hose 30 by supporting and delimiting it laterally (see FIG. 3) and from below, as for example, shown in FIG. 6 in more detail.

The rotor 10 of FIG. 2a and FIG. 2b lies optionally under a cover element 5, which may protect the user from accidentally touching rotor components. The cover element optionally also serves as a handle for manual threading of the hose 30.

The cover element 5 includes or consists of optionally two guiding lugs 4. The guiding lugs 4 may be designed to facilitate the threading of hose 30 by their geometry (see FIG. 3).

The arrow denoted with the reference numeral A illustrates the view direction according to FIG. 3 to FIG. 5.

FIG. 3 shows a schematic representation of only the rotor 10 of FIG. 2a and FIG. 2b with a view from below in the direction of arrow A of FIG. 2a.

The occlusion device 1 and the axial guiding element 6 are clearly visible here, as no hose 30 has been inserted yet.

The axial guiding elements 6 may have different geometrical configurations. They may be made of one or more parts. They include at least one middle section 6c and at least one first lateral section 6a projecting radially beyond the middle section 6c. This first lateral section 6a also projects radially over a section of the outer edge of the rotor 10. The axial guiding elements 6 are provided in order to center or align the hose 30. They are configured such that transverse sliding movements or shearing movements of the hose 30 are prevented or minimized while the rotor 10 is running. In order to further minimize the frictional forces which are exerted on the hose 30, the axial guiding element 6, as in this embodiment, may consist of or include passively rotating parts or rollers.

Only the first lateral section 6a of the axial guiding element 6 is shown here, which is connected to the middle section 6c, see FIG. 3. An optional second lateral section 6b may be provided, see for example, FIG. 3. It may be connected to the first lateral section 6a by the middle section 6c.

According to the present disclosure, for example, the middle section 6c and/or the first lateral section 6a and/or the second lateral section 6b may each be rotatable about their longitudinal axis (or about a center point in the case of an embodiment with a disc-shaped, first lateral section 6a).

Both the occlusion device 1 and the axial guiding element 6 are here exemplarily arranged at a common swing arm 2.

The occlusion device 1 is optionally cylindrically designed.

The axial guiding element 6 of the present embodiment is one-piece, has a cylindrical middle section 6c, a first lateral section 6a, which is substantially disc-shaped, and a second lateral section 6b, which is substantially conical.

The middle section 6c here also has, by way of example, a smaller maximum diameter than the lateral sections 6a and 6b. During use, the axial guiding element 6 of FIG. 3 rotates about its central longitudinal axis L.

In some embodiments, the axial guiding element 6 rotates passively, i.e. its axis or shaft is not mechanically connected to the rotor axis. A rotational movement of the axial guiding element 6 about its longitudinal axis is determined only by the friction against the hose 30, without further forces playing a role for this rotational movement of the axial guiding element 6.

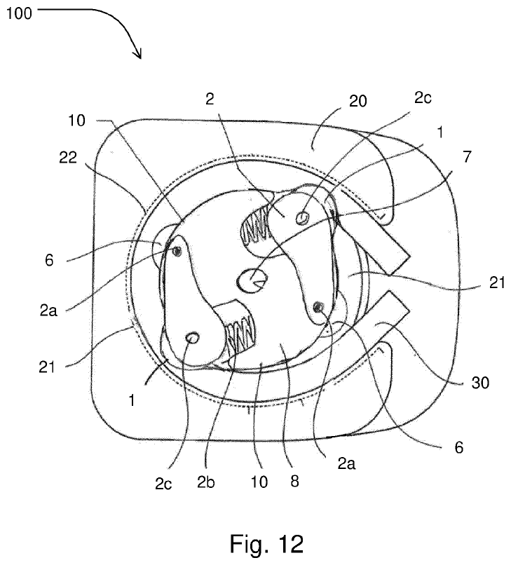

Each of the axial guiding elements 6 may be attached individually to a specific swing arm 2. The axial guiding elements 6 may alternatively be attached in pairs or in groups to a respective common swing arm 2. Alternatively or in addition, at least one of the axial guiding elements 6 may be arranged on a common swing arm 2 with at least one of the occlusion devices 1 (see, for example, the exemplary embodiment according to the present disclosure of FIG. 12).

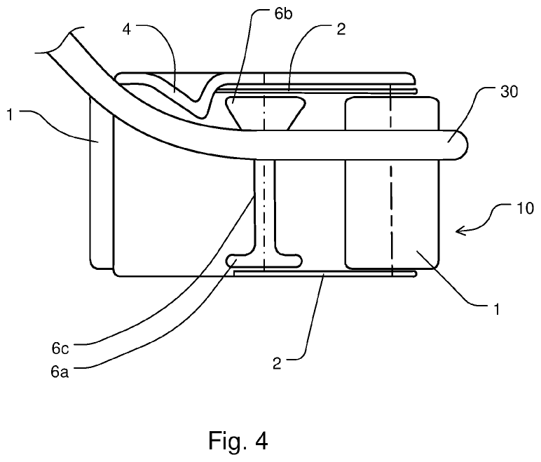

FIG. 4 shows a schematic representation of the rotor 10 during the insertion of a hose 30 into the conveying device 100 with the aid of the guiding lug 4.

In this, the guiding lug 4 (generally: guiding element) is designed to insert, using the axial guiding element 6, the hose 30 between the rotor 10 and the stator 20 simply and with only a small mechanical load.

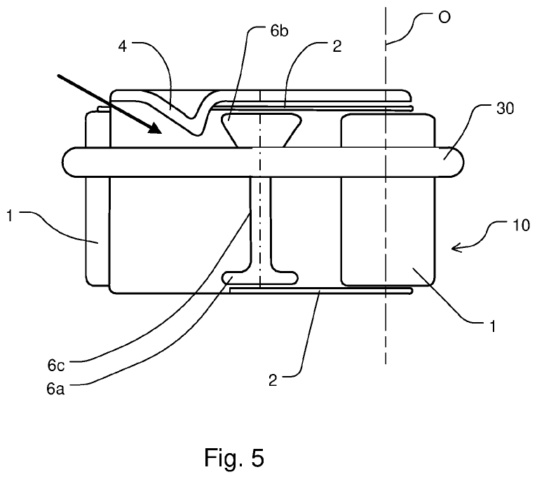

FIG. 5 shows the schematic representation of rotor 10 of FIG. 4 with the hose 30 already inserted.

The geometric design of the guiding element 6 is here exemplarily such that the hose 30 does not come into contact with the guiding lug 4 when the rotor 10 is running. This is illustrated in FIG. 5 by a thin arrow.

FIG. 5 shows how the hose 30 is guided over the middle section 6c and in contact therewith and is simultaneously laterally limited by the first lateral section 6a and the second lateral section 6b of the axial guiding element 6.

The geometry of the axial guiding element 6 keeps the hose centered around the rotor 10 in the stator base 21 such that the hose 30 does not come into contact with the stator base 21, the guiding lug 4 and/or the cover element 5 when the rotor 10 is running.

In this way, the hose 30 inserted between the occlusion devices 1 and the stator 20 advantageously has only a slight lateral or axial play, i.e. the hose 30 is hardly exposed to transverse movements with the rotor running and hardly any shear movements. The reduction in hose abrasion may save material.

FIG. 6 shows a greatly simplified sectional view of a section of the stator 20 with a section of hose 30 inserted between stator 20 and rotor 10 and an axial guiding element 6 in detail.

The service life of the hose 30 may be significantly increased by suitable selection of the construction parameters of the axial guiding element 6. In some embodiments, more constant flow values may also be maintained along the hose 30.

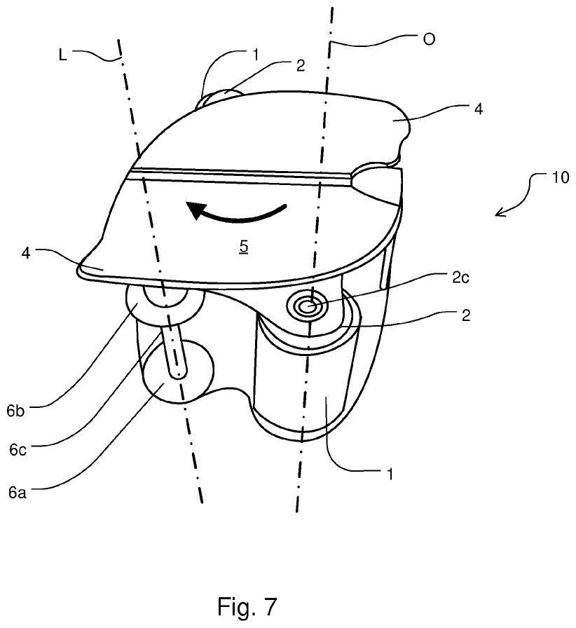

FIG. 7 shows a schematic representation of the rotor 10 of the previous figures in perspective with a view from obliquely below and from the front (outside) to the area in which the hose 30 may be guided around the rotor 10.

Here, the rotor 10 includes the optional cover element 5, which substantially covers the rotor 10. In the radial direction, only the swing arm 2 with the occlusion devices 1 and the axial guiding elements 6 protrude beyond the cover element 5 of the rotor 10. The axis 2c of the occlusion device 1, designed as a swing axis, with which it is mounted in or at the swing arm 2, is clearly visible. This axis 2c allows rotation of the occlusion device 1 about its central longitudinal axis O.

In this embodiment, the two lateral sections 6a and 6b of the axial guiding element 6 are designed, for example, in the form of discs. They optionally have the same diameter. The middle section 6c is cylindrical and has a much smaller diameter than the two lateral sections 6a, 6b. Both lateral sections 6a, 6b may optionally be conical, wherein the respective cone height of the lateral sections 6a, 6b may be the same in some embodiments and different in others.

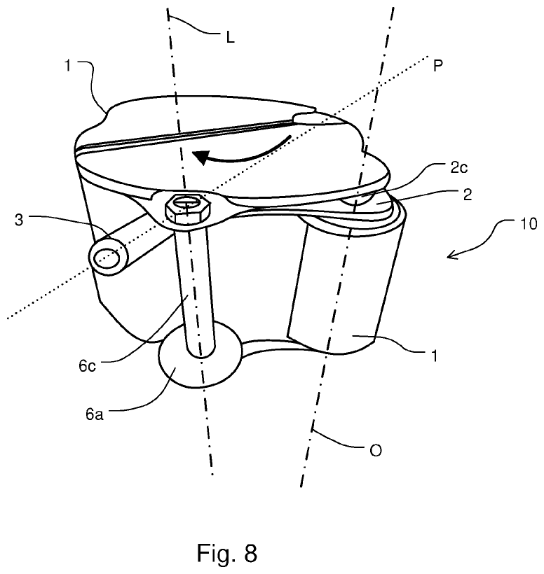

FIG. 8 shows a representation of a further embodiment of the conveying device with a view to the rotor 10 in perspective with an oblique view from below and from the front. The occlusion device 1, an axial guiding element 6 and a lateral guiding element 3 may be easily recognized.

In some embodiments, for example, that of FIG. 8, the axial guiding element 6 includes only the first lateral section 6a and the middle section 6c, but not the second lateral section 6b.

The first lateral section 6a delimits the hose 30 towards the stator base 21. It has a geometric design, for example (preferably completely or substantially) disc-shaped or conical, which is suitable for keeping the hose 30 centered while the rotor 10 is running such that there is no unintentional contact with the stator base 21 or with parts or areas of the rotor body (for example, with the hub body 8).

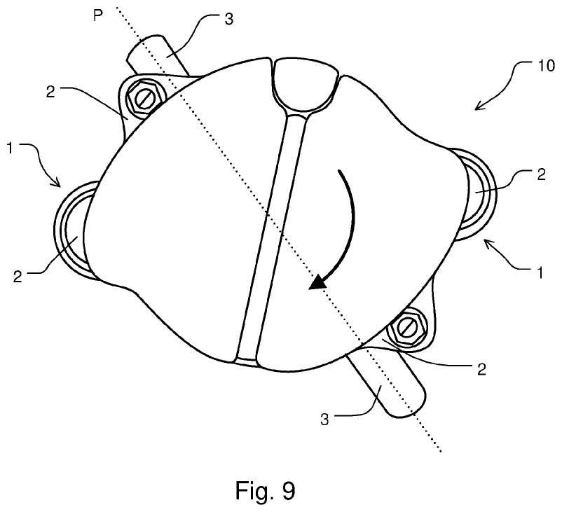

Furthermore, the rotor 10 in this embodiment includes at least one lateral guiding element 3 (see also FIG. 9). The lateral guiding elements 3, of which only one can be seen in FIG. 8, delimit the hose 30 laterally towards the outside of the rotor 10, i.e. to the open side of the space between rotor 10 and stator 20, i.e. away from the stator base 21 or towards the user. Thus, they prevent the hose 30 from sliding out of the conveying device 100 while the rotor 10 is running. In interaction with the axial guiding element 6, the lateral guiding elements 3 prevent transverse sliding or shearing movements of the hose 30 from occurring.

The at least one lateral guiding element 3 may be designed as a cylindrical or substantially cylindrical body or may have one which is arranged on an axis through one of its base surfaces, the shaft extending radially to the rotor shaft 7. The longitudinal axis of the lateral guiding element 3 is preferably perpendicular to the longitudinal axis of the rotor shaft 7. The lateral guiding element 3 may optionally rotate about its longitudinal axis P in order to reduce the frictional forces against the hose 30 when the rotor is running.

FIG. 9 shows the rotor 10 of FIG. 8 in a front view.

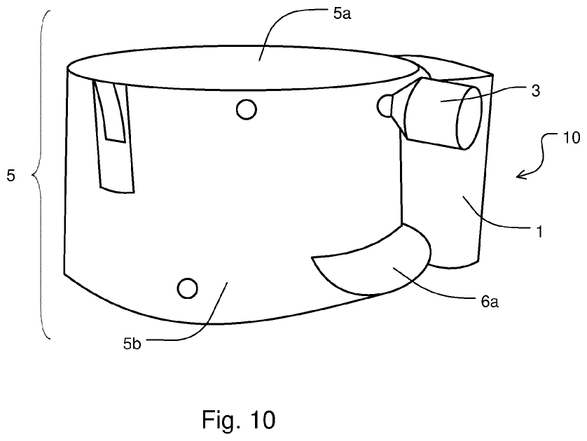

FIG. 10 shows a section of again a further embodiment of the conveying device 100 with a view to the rotor 10 in a slight perspective from below.

Here, the cover element 5 forms a housing with an upper surface or surface 5a which covers the rotor 10 outwardly towards the user and which includes the guiding lugs 4, and with a shell or border 5b which optionally extends to or near to the stator base 21 and which has recesses through which the occlusion devices 1, the axis of the at least one lateral guiding element 3 and/or lateral sections of the at least one axial guiding element 6, or sections thereof, may protrude beyond the rotor 10.

For example, in this embodiment the middle section 6c of the axial guiding element 6 is integrated in the housing. The first lateral section 6a of the axial guiding element 6, which is designed in the form of a disc, is exemplarily configured here as a rotating part and may clearly protrude laterally beyond the cover element 5.

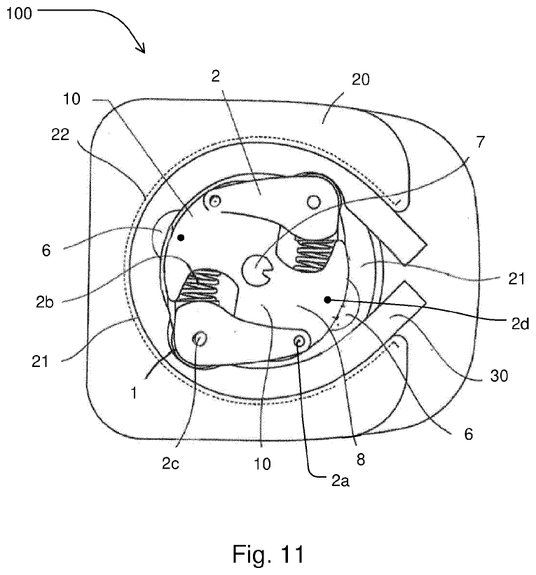

FIG. 11 shows a schematic representation of a further embodiment of the conveying device 100 in a slight perspective from the front with a view to the swing arm(s) 2.

The swing arm 2 may have any design known in the prior art. For example, it may be designed as a fork with a swinging joint 2a, which connects the fork to a hub body 8. Furthermore, it may be designed with a swinging spring 2b, as well as with an axis 2c, in order to attach the occlusion device 1 to the fork of the swing arm 2 in a freely rotating manner.

The hub body 8 couples the swing arm 2 and thus all parts arranged on it to the rotor 10, which is driven by a shaft of a drive motor of the blood treatment apparatus, not shown. In these embodiments, the hub body 8 couples, for example, all components of the rotor 10 which extend radially in the rotor 10, such as the occlusion devices 1 and axial guiding elements 6 by an axis 2d, to the rotation of same.

In the embodiment shown in FIG. 11, the axial guiding elements 6 are not arranged on one of the swing arms 2.

The swing arm(s) 2 may be rigid structures or slightly springy (as shown in FIGS. 11 and 12). A slight springing of the swing arm(s) may, for example, be advantageous to allow the rotor 10 to adapt to different hoses, for example, hoses with different diameters.

In principle, different designs of the swing arms 2 in a single embodiment of the rotor 10 are also encompassed by the present disclosure.

FIG. 12 shows a schematic representation of a further embodiment of the conveying device 100 according to the present disclosure with a swing arm 2 and without a cover element, viewed from the front.

The structure of the present embodiment also largely corresponds to the design of the embodiment in FIG. 11. However, here at least its occlusion device 1 is connected to an axial guiding element 6 by a swing arm 2, wherein the swinging joint 2a serves as an axis for the axial guiding element 6. At least one occlusion device 1 and at least one axial guiding element 6 are thus present on a common swing arm 2.

The occlusion devices 1 may also be spring-mounted by the swinging springs 2b, which may facilitate the insertion of a hose 30 into the rotor 10 and/or may facilitate the adaptation of the rotor 10 to different hoses, as described herein.

The statements given above for FIG. 11, such as for the hub body 8, also apply undiminished to the embodiments or representations in FIG. 12.

### LIST OF REFERENCE NUMERALS

1 occlusion device
2 swing arm
2a swinging joint
2b swinging spring
2c axis of the occlusion device
2d axis of the guiding element
3 lateral guiding element
4 guiding lug
5a surface, upper surface
5b border, shell
5 cover element
6 axial guiding element
6a first lateral section towards the machine side of the axial guiding element
6b second lateral section towards the user side of the axial guiding element
6c middle section of the axial guiding element
7 drive shaft, or rotor shaft or axis
8 hub body
10 rotor
20 stator
21 stator base
22 hose bed as a counter bearing for the occlusion devices
30 hose
100 conveying device
L central longitudinal axis of the axial guiding element
Q central transverse axis of the axial guiding element
O central longitudinal axis of the occlusion device
P central longitudinal axis of the lateral guiding element

1-15. (canceled)

16. A conveying device for conveying a medical fluid that is guided into a hose, the conveying device comprising:
a rotor; and
a stator comprising:
a stator base,
an area for receiving the hose in the conveying device, and
a hose bed providing a counter bearing for occlusion devices,
wherein the rotor comprises a rotor axis and at least two occlusion devices that are attached radially to the rotor axis and that compress the hose intermittently against the hose bed during use of the conveying device,
wherein the rotor encompasses an axial guiding element that is arranged to rotate about its own longitudinal axis for aligning the hose in the stator,
wherein the longitudinal axis of the axial guiding element extends parallel to the longitudinal axis of the rotor axis,
wherein the axial guiding element comprises a middle section and a first lateral section protruding radially over the middle section,
wherein the first lateral section is rotatably arranged, and
wherein part of the first lateral section protrudes radially over a section of an outer edge of the rotor.

17. The conveying device according to claim 16, wherein the axial guiding element is a first axial guiding element, and wherein the rotor further comprises a second axial guiding element, wherein at least one of the first and second axial guiding elements or at least one of the at least two occlusion devices is arranged on a swing arm of the rotor.

18. The conveying device according to claim 17, wherein the first and second axial guiding elements and/or the at least two occlusion devices are arranged opposite to each other on a common swing arm of the rotor.

19. The conveying device according to claim 16, wherein the rotor comprises a plurality of occlusion devices and a plurality of axial guiding elements that are arranged on a swing arm such that the plurality of axial guiding elements and the plurality of occlusion devices are arranged alternately in the rotor.

20. The conveying device according to claim 19, wherein at least one axial guiding element of the plurality of axial guiding elements comprises a second lateral section.

21. The conveying device according to claim 20, wherein the first and the second lateral sections of the at least one axial guiding element are conical and together form a double cone, wherein the middle section of the at least one axial guiding element is arranged between the first and the second lateral sections and connects them to each other.

22. The conveying device according to claim 21, wherein the first and second lateral sections are shaped differently from each other such that the double cone is not point-symmetrical to its geometric center of gravity.

23. The conveying device according to claim 16, wherein the rotor further comprises a lateral guiding element having its own longitudinal axis that extends perpendicular to the rotor axis that is arranged opposite to the stator base, and that limits a direction of the hose towards only a front or towards only an outside.

24. The conveying device according to claim 23, wherein the lateral guiding element is arranged rotatably about its own longitudinal axis.

25. The conveying device according to claim 16, wherein only the middle section of the axial guiding element is rotatable about its own longitudinal axis.

26. The conveying device according to claim 16, further comprising a cover element that at least partially covers the rotor.

27. The conveying device according to claim 26, wherein the cover element comprises at least one guiding lug for easy and safe insertion of the hose in the area between rotor and stator.

28. The conveying device according to claim 26, wherein at least one axial guiding element and/or one lateral guiding element is integrated into the cover element.

29. A blood treatment apparatus comprising a conveying device, the conveying device comprising:
a rotor; and
a stator comprising:
a stator base,
an area for receiving the hose in the conveying device, and
a hose bed providing a counter bearing for occlusion devices,
wherein the rotor comprises a rotor axis and at least two occlusion devices that are attached radially to the rotor axis and that compress the hose intermittently against the hose bed during use of the conveying device,
wherein the rotor encompasses an axial guiding element that is arranged to rotate about its own longitudinal axis for aligning the hose in the stator,
wherein the longitudinal axis of the axial guiding element extends parallel to the longitudinal axis of the rotor axis,
wherein the axial guiding element comprises a middle section and a first lateral section protruding radially over the middle section,
wherein the first lateral section is rotatably arranged, and
wherein part of the first lateral section protrudes radially over a section of an outer edge of the rotor.

30. The blood treatment apparatus according to claim 29, wherein the blood treatment apparatus comprises one or more of a dialysis apparatus, a hemodialysis apparatus, a hemofiltration apparatus, a hemodiafiltration apparatus, an apheresis apparatus, a plasma treatment or plasma exchange apparatus, and a therapeutic plasma exchange apparatus.

31. The blood treatment apparatus according to claim 29, wherein the axial guiding element is a first axial guiding element, and wherein the rotor further comprises a second axial guiding element, wherein at least one of the first and second axial guiding elements or at least one of the at least two occlusion devices is arranged on a swing arm of the rotor.

32. The blood treatment apparatus according to claim 31, wherein the first and second axial guiding elements and/or the at least two occlusion devices are arranged opposite to each other on a common swing arm of the rotor.

33. The blood treatment apparatus according to claim 29, wherein the rotor comprises a plurality of occlusion devices and a plurality of axial guiding elements that are arranged on a swing arm such that the plurality of axial guiding elements and the plurality of occlusion devices are arranged alternately in the rotor.

34. The blood treatment apparatus according to claim 33, wherein at least one axial guiding element of the plurality of axial guiding elements comprises a second lateral section.

35. The blood treatment apparatus according to claim 34, wherein the first and the second lateral sections of the at least one axial guiding element are conical and together form a double cone, wherein the middle section of the at least one axial guiding element is arranged between the first and the second lateral sections and connects them to each other.
